(19) United States
(12) Patent Application Publication
(10) Pub. No.: US 2023/0187035 A1
(43) Pub. Date: Jun. 15, 2023

(54) METHOD AND APPARATUS FOR PEOPLE HEALTH

(71) Applicant: Telefonaktiebolaget LM Ericsson (publ), Stockholm (SE)

(72) Inventor: Ning ZHANG, Beijing (CN)

(73) Assignee: Telefonaktiebolaget LM Ericsson (publ), Stockholm (SE)

(21) Appl. No.: 17/916,689

(22) PCT Filed: Apr. 3, 2020
(86) PCT No.: PCT/CN2020/083235

§ 371 Date: Oct. 3, 2022

(51) Int. Cl. G16H 10/60; G16H 20/00; G16H 50/20; G16H 80/00

(52) U.S. Cl. CPC G16H 10/60; G16H 20/00; G16H 50/20; G16H 80/00

(57) ABSTRACT

Embodiments of the present disclosure provide methods and apparatuses for people health. A method at a first communication device comprises receiving information related to a person during a health care procedure of the person. The method further comprises generating feedback information related to the person based on the information related to the person. The method further comprises sending the feedback information related to the person to a terminal device of the person.

### TECHNICAL FIELD

The non-limiting and exemplary embodiments of the present disclosure generally relate to the technical field of communications, and specifically to methods and apparatuses for people health.

### BACKGROUND

This section introduces aspects that may facilitate a better understanding of the disclosure. Accordingly, the statements of this section are to be read in this light and are not to be understood as admissions about what is in the prior art or what is not in the prior art.

Currently, medical resources may be limited in some areas, let alone health rehabilitation and/or recovery assistance. Because of lacking sufficient sustainable, suitable and specified treatment, recovery and rehabilitation after therapy, many persons' rehabilitation and/or recovery procedures are rather long which may result in time wasting, and a worse situation may be that they do not know they are in a wrong method of health rehabilitation and/or recovery so that their health status may be getting deterioration. This may be tend to happen for example in remote and backward areas where people are less educated.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter.

Usually, after therapy for example in a hospital, the person such as a patient may be advised by a health specialist such as doctor for a consequence health care procedure. However, each person has his/her own situation, the health specialist can't evaluate which health method is suitable for the person for example due to tight inquiry and therapy schedule. In addition, the person also does not know how to report the person's living habit and the health status, the person might also hide some habits and customs intentionally or unintentionally. This would increase the difficulty of the health specialist's further therapy or health for the person. Also, when an athlete participates strenuous exercise or a series of competition, the recovery from exercise fatigue is the prioritized task for this athlete. These methods and apparatuses can also be applied to athlete and their coaches, health consultant and nutritionist.

Even some advanced hospitals have various medical records from which it would be easier to find out a quiet similar person so that it may help the person to fulfill the health care procedure to recover soon, and the person acknowledges as well, but in practice, the person does not realize that he/she does not follow the health care procedure. Thus, when the person is re-exanimated in the hospital, the person's health status may not be improved. The health specialist might be confused in the person's health care procedure which may impact the health care procedure significantly.

To overcome or mitigate at least one of the above mentioned problems or other problems, embodiments of the present disclosure propose an improved health solution.

In a first aspect of the disclosure, there is provided a method at a first communication device. The method comprises receiving information related to a person during a health care procedure of the person. The method further comprises generating feedback information related to the person based on the information related to the person. The method further comprises sending the feedback information related to the person to a terminal device of the person.

In an embodiment, the feedback information related to the person may be authorized by a health specialist.

In an embodiment, generating feedback information related to the person based on the information related to the person may comprise providing the information related to the person to a health specialist; and receiving the feedback information related to the person input by the health specialist.

In an embodiment, generating feedback information related to the person based on the information related to the person may comprise generating preliminary feedback information related to the person based on the information related to the person; providing the preliminary feedback information related to the person to a health specialist; and receiving the feedback information related to the person input by the health specialist.

In an embodiment, the feedback information related to the person may include at least one of a health level of the person; a suggestion; and an updated health plan.

In an embodiment, generating feedback information related to the person based on the information related to the person may comprise comparing current information related to the person with previous information related to the person; and determining the health level of the person based on the comparing result.

In an embodiment, generating feedback information related to the person based on the information related to the person may comprise determining one or more factors having a strong relationship of the health care procedure based on the information related to at least one quickest recovering person, wherein the at least one quickest recovering person and the person belong to a same or similar health group; determining one or more gaps between the person and the quickest recovering person based on the one or more factors having the strong relationship of the health care procedure and/or an ideal recovering model by historical data; and generating the suggestion based on the one or more gaps.

In an embodiment, the information related to the person may include at least one of a micro expression video clip of the person; a series of body gestures of the person; a shopping record of the person; a sport activity plan of the person; a sport activity record of the person; a health status of the person; a body physical status of the person; a medical record of the person; a work habit of the person; a life habit of the person; diet information of the person; a physical status of the person; a psychological status of the person; nutrition information of the person; and rest information of the person.

In an embodiment, when there are two or more types of the information related to the person, at least one of the two or more types of the information related to the person may be configured with a weight value.

In an embodiment, the method may further comprise sending the information related to the person to a second communication device which can be accessed by a medical institution.

In an embodiment, the method may further comprise receiving an initial health plan related to the person from a second communication device which can be accessed by a medical institution; and sending the initial health plan related to the person to the terminal device of the person.

In an embodiment, the health care procedure may comprise at least one of health rehabilitation; health recovery; improvement of health; and strengthening of a level of athletes' competition.

In a second aspect of the disclosure, there is provided a method at a third communication device. The method comprises obtaining information related to a person during a health care procedure of the person. The method further comprises sending, to a first communication device, the information related to the person.

In an embodiment, the method may further comprise receiving, from the communication device, feedback information related to the person.

In an embodiment, the method may further comprise receiving an initial health plan related to the person from the first communication device.

In a third aspect of the disclosure, there is provided a method at a second communication device. The method comprises receiving, from a first communication device, information related to at least one person belonging to a same or similar health group during a health care procedure of the at least one person. The method further comprises determining a health plan for the same or similar health group based on the information related to at least one person.

In an embodiment, the information related to the at least one person may include at least one of a micro expression video clip of the at least one person; a series of body gestures of the at least one person; a shopping record of the at least one person; a sport activity plan of the at least one person; a sport activity record of the at least one person; a health status of the at least one person; a body physical status of the at least one person; a medical record of the at least one person; a work habit of the at least one person; a life habit of the at least one person; diet information of the at least one person; a physical status of the at least one person; a psychological status of the at least one person; nutrition information of the at least one person; and rest information of the at least one person.

In an embodiment, when there are two or more types of the information related to the at least one person, at least one of the two or more types of the information related to the at least one person may be configured with a weight value.

In an embodiment, the method may further comprises sending the health plan for the same or similar health group to the first communication device.

In a fourth aspect of the disclosure, there is provided a first communication device. The first communication device comprises a processor; and a memory coupled to the processor, said memory containing instructions executable by said processor, whereby said first communication device is operative to receive information related to a person during a health care procedure of the person. Said first communication device is further operative to generate feedback information related to the person based on the information related to the person. Said first communication device is further operative to send the feedback information related to the person to a terminal device of the person.

In a fifth aspect of the disclosure, there is provided a third communication device. The third communication device comprises a processor; and a memory coupled to the processor, said memory containing instructions executable by said processor, whereby said third communication device is operative to obtain information related to a person during a health care procedure of the person. Said third communication device is further operative to send, to a first communication device, the information related to the person.

In a sixth aspect of the disclosure, there is provided a second communication device. The second communication device comprises a processor; and a memory coupled to the processor, said memory containing instructions executable by said processor, whereby said second communication device is operative to receive, from a first communication device, information related to at least one person belonging to a same or similar health group during a health care procedure of the at least one person. Said second communication device is further operative to determine a health plan for the same or similar health group based on the information related to at least one person.

In a seventh aspect of the disclosure, there is provided a computer-readable storage medium storing instructions which when executed by at least one processor, cause the at least one processor to perform the method according to any one of the first, second and third aspects of the disclosure.

In an eighth aspect of the disclosure, there is provided a computer program product comprising instructions which when executed by at least one processor, cause the at least one processor to perform the method according to any one of the first, second and third aspects of the disclosure.

In a ninth aspect of the disclosure, there is provided a first communication device. The first communication device comprises a receiving module, a generating module and a sending module. The receiving module may be configured to receive information related to a person during a health care procedure of the person. The generating module may be configured to generate feedback information related to the person based on the information related to the person. The sending module may be configured to send the feedback information related to the person to a terminal device of the person.

In a tenth aspect of the disclosure, there is provided a third communication device. The third communication device comprises a obtaining module and a sending module. The obtaining module may be configured to obtain information related to a person during a health care procedure of the person. The sending module may be configured to send, to a first communication device, the information related to the person.

In an eleventh aspect of the disclosure, there is provided a second communication device. The second communication device comprises a receiving module and a determining module. The receiving module may be configured to receive, from a first communication device, information related to at least one person belonging to a same or similar health group during a health care procedure of the at least one person. The determining module may be configured to determine a health plan for the same or similar health group based on the information related to at least one person.

Embodiments herein afford many advantages, of which a non-exhaustive list of examples follows. Some embodiments herein can improve the person's health care procedure. For example, the person may not be suffered too long if the person's health care procedure is in a wrong way (which may happen in some areas such as remote and backward areas). Some embodiments herein can enable the health specialist's efforts on the consequence treatment based on the health status to be significantly reduced. Some embodiments herein can enable the health specialist to pay more efforts on study more cases or improve the medical professional skills that may bring much more benefit to the person who requires health care. In some embodiments herein, the information between the person and the health specialist is more transparent and objective with less interference information. Some embodiments herein can utilize various facilities such as camera along the street, video captured by phones, online shopping records, sports exercises plans and records, etc. to create a database for the person who requires health care. The embodiments herein are not limited to the features and advantages mentioned above. A person skilled in the art will recognize additional features and advantages upon reading the following detailed description.

### BRIEF DESCRIPTION OF THE DRAWINGS

The above and other aspects, features, and benefits of various embodiments of the present disclosure will become more fully apparent, by way of example, from the following detailed description with reference to the accompanying drawings, in which like reference numerals or letters are used to designate like or equivalent elements. The drawings are illustrated for facilitating better understanding of the embodiments of the disclosure and not necessarily drawn to scale, in which:

FIG. 1 depicts a schematic system, in which some embodiments of the present disclosure can be implemented;

FIG. 2 depicts a diagram of how the system works as combined from the person, the health specialist and the medical institution according to an embodiment of the present disclosure;

FIG. 3 depicts a diagram of person data collection according to an embodiment of the present disclosure

FIG. 4 shows a flowchart of a method according to an embodiment of the present disclosure;

FIG. 5 shows a flowchart of a method according to another embodiment of the present disclosure;

FIG. 6 shows a flowchart of a method according to another embodiment of the present disclosure;

FIG. 7 shows a flowchart of a method according to another embodiment of the present disclosure;

FIG. 8 is a block diagram showing an apparatus suitable for practicing some embodiments of the disclosure;

FIG. 9 is a block diagram showing a first communication device according to an embodiment of the disclosure;

FIG. 10 is a block diagram showing a third communication device according to an embodiment of the disclosure; and

FIG. 11 is a block diagram showing a second communication device according to an embodiment of the disclosure.

### DETAILED DESCRIPTION

The embodiments of the present disclosure are described in detail with reference to the accompanying drawings. It should be understood that these embodiments are discussed only for the purpose of enabling those skilled persons in the art to better understand and thus implement the present disclosure, rather than suggesting any limitations on the scope of the present disclosure. Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Furthermore, the described features, advantages, and characteristics of the disclosure may be combined in any suitable manner in one or more embodiments. One skilled in the relevant art will recognize that the disclosure may be practiced without one or more of the specific features or advantages of a particular embodiment. In other instances, additional features and advantages may be recognized in certain embodiments that may not be present in all embodiments of the disclosure.

As used herein, the term “network” refers to a network following any suitable (wireless or wired) communication standards. For example, the wireless communication standards may comprise new radio (NR), long term evolution (LTE), LTE-Advanced, wideband code division multiple access (WCDMA), high-speed packet access (HSPA), Code Division Multiple Access (CDMA), Time Division Multiple Address (TDMA), Frequency Division Multiple Access (FDMA), Orthogonal Frequency-Division Multiple Access (OFDMA), Single carrier frequency division multiple access (SC-FDMA) and other wireless networks. A CDMA network may implement a radio technology such as Universal Terrestrial Radio Access (UTRA), etc. UTRA includes WCDMA and other variants of CDMA. A TDMA network may implement a radio technology such as Global System for Mobile Communications (GSM). An OFDMA network may implement a radio technology such as Evolved UTRA (E-UTRA), Ultra Mobile Broadband (UMB), IEEE 802.11 (Wi-Fi), IEEE 802.16 (WiMAX), IEEE 802.20, Flash-OFDMA, Ad-hoc network, wireless sensor network, etc. In the following description, the terms “network” and “system” can be used interchangeably. Furthermore, the communications between two devices in the network may be performed according to any suitable communication protocols, including, but not limited to, the wireless communication protocols as defined by a standard organization such as 3rd generation partnership project (3GPP) or the wired communication protocols. For example, the wireless communication protocols may comprise the first generation (1G), 2G, 3G, 4G, 4.5G, 5G communication protocols, and/or any other protocols either currently known or to be developed in the future.

As used herein, the term “communication device” refers to any electronic equipment that implements communication function. The communication device may operably communicate with terminal devices through the network. The communication device may provide various services such as medical services or health services. The communication device can be implemented in form of hardware, software or their combination, including but not limited to, terminal device, medical device, municipal facility, cloud computer, distributed communication system, virtual computer, server, personal computer, desktop computer, etc. The communication device may run with any kind of operating system including, but not limited to, Windows, Linux, UNIX, Android, iOS and their variants.

As used herein, the term “terminal device” refers to any end device that can access a communication network and receive services therefrom. By way of example and not limitation, in the wireless communication network, the terminal device may refer to a mobile terminal, a user equipment (UE), a terminal device, or other suitable devices. The terminal device may be, for example, a Subscriber Station (SS), a Portable Subscriber Station, a Mobile Station (MS), or an Access Terminal (AT). The terminal device may include, but not limited to, a portable computer, an image capture device such as a digital camera, a gaming terminal device, a music storage and a playback appliance, a mobile phone, a cellular phone, a smart phone, a voice over IP (VoIP) phone, a wireless local loop phone, a tablet, a wearable device, a personal digital assistant (PDA), a portable computer, a desktop computer, a wearable device, a vehicle-mounted wireless device, a wireless endpoint, a mobile station, a laptop-embedded equipment (LEE), a laptop-mounted equipment (LME), a Universal Serial Bus (USB) dongle, a smart device, a wireless customer-premises equipment (CPE) and the like. In the following description, the terms “terminal device”, “terminal”, “user equipment” and “UE” may be used interchangeably. As one example, a UE may represent a terminal device configured for communication in accordance with one or more communication standards promulgated by the 3GPP, such as LTE standard or NR standard. As used herein, a “user equipment” or “UE” may not necessarily have a “user” in the sense of a human user who owns and/or operates the relevant device. In some embodiments, a terminal device may be configured to transmit and/or receive information without direct human interaction. For instance, a UE may be designed to transmit information to a network on a predetermined schedule, when triggered by an internal or external event, or in response to requests from the wireless communication network. Instead, a UE may represent a device that is intended for sale to, or operation by, a human user but that may not initially be associated with a specific human user.

As yet another example, in an Internet of Things (IoT) scenario, a terminal device may represent a machine or other device that can perform monitoring and/or measurements, and transmit the results of such monitoring and/or measurements to another terminal device and/or network equipment. The terminal device may in this case be a machine-to-machine (M2M) device, which may in a 3GPP context be referred to as a machine-type communication (MTC) device. As one particular example, the terminal device may be a UE implementing the 3GPP narrow band internet of things (NB-IoT) standard. Particular examples of such machines or devices are sensors, metering devices such as power meters, industrial machinery, or home or personal appliances, for example refrigerators, televisions, personal wearables such as watches etc. In other scenarios, a terminal device may represent a vehicle or other equipment that is capable of monitoring and/or reporting on its operational status or other functions associated with its operation.

References in the specification to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the associated listed terms.

As used herein, the phrase “at least one of A and B” should be understood to mean “only A, only B, or both A and B.” The phrase “A and/or B” should be understood to mean “only A, only B, or both A and B.”

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “has”, and/or “includes”, when used herein, specify the presence of stated features, elements, and/or components etc., but do not preclude the presence or addition of one or more other features, elements, components and/ or combinations thereof.

It is noted that these terms as used in this document are used only for ease of description and differentiation among nodes, devices or networks etc. With the development of the technology, other terms with the similar/same meanings may also be used.

In the following description and claims, unless defined otherwise, all technical and scientific terms used herein have the same meaning as commonly understood by one of ordinary skills in the art to which this disclosure belongs.

Currently, there are many facilities that are equipped but without information sharing. For example, there are many cameras that are installed along a street as a key fundamental factor of public security. However, due to a privacy policy, those data such as face images, the gesture of walking, etc. can be only shared with a public security bureaus. Many mobile phones are becoming even popular and video clips may be stored on a cloud storage, however due to the privacy, they are only shared among family members and familiar peoples. There are various online shopping records. Existing solutions provide some further suggestions based on the previous purchased goods. However, they are always turned out to be a series advertisement which is not so friendly to people. Sports activities plans and records somehow just mean to “showcase” in social networks, that is not what they are aim to. If these data can be shared, it would bring a benefit to the person. When the person is cured from a disease, a prioritized task for the person is health such as rehabilitation. If the person agrees to share the information that relevant to health status to hospital or any other medical institution for analysis, then it may bring a significant benefit in the health care procedure.

Some embodiments herein may be implemented in some perspective: the person (such as patient) requiring health, the health specialist (such as doctor) and the medical institution.

For the person requiring health:

a. In some areas such as developing countries, the medical resources (for example, the doctor and the medical equipment) are rather limited, the person can not have sufficient time to communicate with the health specialist such as doctor and discuss the health care procedure. In some cases, the situation is even worse. For example, the person can not even get a registration to see a doctor. This experience may be bad and may be a major factor which may affect the health care procedure. The person may become very proactive to wait in a queue before seeing a doctor both in the real world or online. There can be specified wearable medical devices for the person, but the person might have a bad feeling that the person is being observed.

b. The person can free his/her time to think up all these annoying steps as a preparation to discuss with a health specialist, the information may be collected and shared to the health specialist in below ways:

i. The person just needs to wear glasses as usual that are equipped with a first camera which can capture a view when seeing, a second camera that can log his/her micro expressions, and a gyroscope which can record a pattern when walking.

ii. Then, the data captured in above step (i) may be transferred to a database that is owned by a hospital(s) and/or other medical institution(s) under a permission of the person.

iii. The captured data may be analyzed and the result and further health plan(s) may be provided to his/her health specialist, and finally the health specialist may review and adjust the health plan that make it more suitable to this person.

For the health specialist:

a. The advantage for a health specialist may be that the health specialist may have a chance to get various data related to the person so long as the person agrees, then with the data collected from each person, it's easier to get some patterns for health care procedure refinement.

b. Currently, the health specialist has very limited ways to know his/her professional level among a certain area. Based on all these medical records that cured by the health specialist, it can also help the health specialist to get a baseline to all persons' health status, and in comparison with other health specialists, the health specialist can know his/her professional skill level.

For the medical institution:

a. Hospitals in some areas seldom share the person's medical records in order to protect the person's privacy. However, if the person's medical records can be shared within doctors and medical institutions with necessary privacy protection, the benefit would be significant to both persons and medical researches.

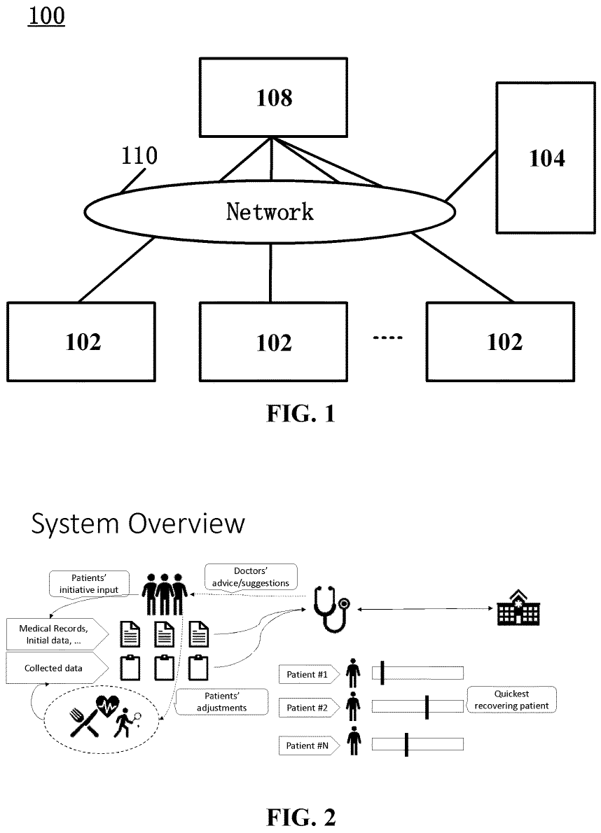

FIG. 1 depicts a schematic system, in which some embodiments of the present disclosure can be implemented. As shown in FIG. 1, the system 100 may comprise one or more third communication devices 102 each operably connected to a first communication device 104 through a network 110. The first communication device 104 can be operably connected to a second communication device 108 through the network 110. Though only one first communication device and only one second communication device are shown in FIG. 1, there can be any suitable number of first and/or second communication device in other embodiments.

FIG. 2 depicts a diagram of how the system works as combined from the person, the health specialist and the medical institution according to an embodiment of the present disclosure. Note that all the steps/operations as shown in FIG. 2 are under the person's permission of sharing his/her information.

As shown in FIG. 2, the person shares his/her work and life habit as well as his/her medical records to the health specialist and medical institutions as an initial step.

Then the person's user device (such as mobile phone) may share some data for example in a daily basis in the entire treatment, recovery and rehabilitation procedure. For example, the data captured by the camera installed in a kitchen and/or glasses with cameras when cooking may be shared. In this way, it can easily know what and how the person eats. For example, does the person have a nutritious balanced meal? Does the meal be cooked in a reasonable way? Alternatively, such information can be fetched from his/her online shopping records in the category of food.

Sports activities records and training plans of the person may be shared. For example, by comparing the items before and after the person's treatment, the health specialist can more objectively know how serious to the person after his/her injury. Also, by referring the training plan and record changes in the health care procedure, the health specialist can understand the progress of how the person is recovered. The records may include walk frequency and step counts and/or the physical status of the person when doing sports. Sometimes the person's exercise plan is not healthy at all, e.g. when doing extensive exercise in a hungry state for a long time.

The above operations may be compared with the person's information in the initial step to correct the wrong data, and they may be run iteratively daily. In this way, it may compare the data that is concerned by health specialists to avoid collecting non-valuable information. After collecting enough information from various persons, the health specialist may do the following steps to make more specified suggestions to each person in the health care procedure: dividing all persons in groups for example by each disease; in each disease, comparing all persons in below ways: finding out the person who recovers most quickly and checking the aforementioned information; checking why other persons recover relatively slow and comparing the information between the quickest recovering person and the slow recovering persons. After the analysis for example by the health specialist, a special suggestion can be sent to the slow recovering persons who may be observed whether they do the change. If they do the change, then the progress of recovery may be checked iteratively. Moreover, all medical institutions such as hospitals, medical colleges can perform more extensively studies and researches on these information.

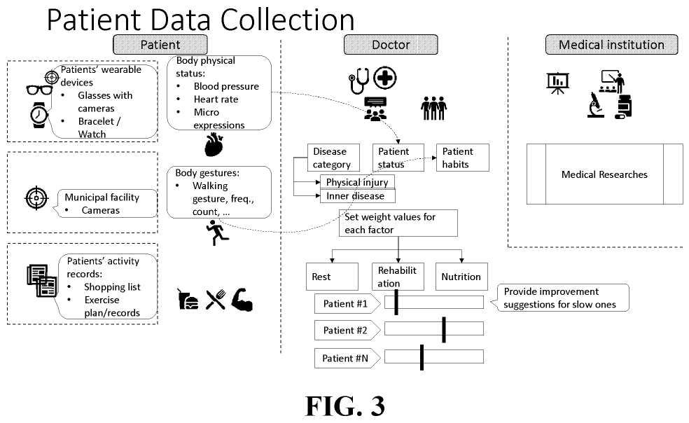

FIG. 3 depicts a diagram of person data collection according to an embodiment of the present disclosure, which illustrates the way that how the data is collected and transferred to the health specialist, how the data is used for medical analysis, and how the parameters of input data from persons may be adjusted.

The terminal device can be below devices (or software applications): glass with cameras, for example, the primacy camera captures the view that the user's seeing while the secondary camera captures the micro express of the person; bracelet that can detect heart rate/pattern, blood pressure and walking frequency/count. If the user's kitchen is installed with a camera, then the video can also be captured.

The data may be online shopping list of foods and/or sport exercise plan and records which can be obtained in various ways. Based on the list of foods, the health specialist can know whether the meals are meet to the requirement of health. Based on the exercise plan and records, the health specialist can know whether this person is in good recovery progress.

Also, the municipal facility can be used as assistant for person data collection. For example, camera installed along the streets are usually owned by the police office and can be shared for medical usage.

The detailed algorithm as the assistant for the health specialist may be as following. A weight value for each type of information related to the person based on the disease of the person may be configured. For example, if the person is suffering from physical injury such as legs or arms injuries, then rest may be a more important factor comparing to nutrition. Also, health by a professional trainer is good to him/her. Thus, in this example, the health specialist or communication device may place more weight value on rest than nutrition. If the person is suffering from disease, then both rest and nutrition may play important roles in his/her health care procedure. Thus, the health specialist or communication device may place similar weight values on both. Also, the quality of rest, such as sleeping, the method that the food's cooked, the person status of physical and psychological may play respective roles in the person health care procedure. For example, sometimes psychological factors can affect the overall recovery procedures significantly even than rest or nutrition.

The health specialist or communication device may collect the data related to the persons in similar basic conditions and compare the difference between the most recovering person and the relatively slow recovering persons. For example, the health specialist or communication device may compare the body gesture and other physical data from camera and health exercise records. The person's body gesture changes can be obtained by a camera installed along streets, especially if the person was suffering physical injuries. By comparing the difference between the current body gesture of the person and the previous days' body gesture of the person, and referring to the correct and normal body gesture, it can make a conclusion that how well this person is recovered.

Also, the person's physical data such as heart rate / pattern, blood pressure and walking frequency/count would be changed (improved or deteriorated), the health specialist or communication device can refer to FIG. 3 and evaluate the recovery progress more accurately.

Moreover, the person's micro express changes, sports exercise plan changes and work schedule changes are supporting evidence for the health specialist's reference.

Then, the health specialist or communication device may compare the slow recovering persons' data with the quickest recovering person's data and provide improvement suggestions for them. For example, based on the information that collected and shared from the quickest one, the health specialist or communication device may select some major factors that have a strong relationship of the health care procedure. The health specialist or communication device may analyze the slow recovering persons' data and find out the facts that have big gaps with the quickest recovering person. The health specialist or communication device may provide suggestions with other medical knowledge and provide feasible adjusted health plans to the slow recovering person.

Moreover, the medical institution can refer the data related to the persons and perform studies and researches on the data to determine the effective health plans. The medical institution can refer to the facts for below areas: the relationship between living habits and a certain disease and/or the health care procedure pattern in different areas (cities) and the improvement space.

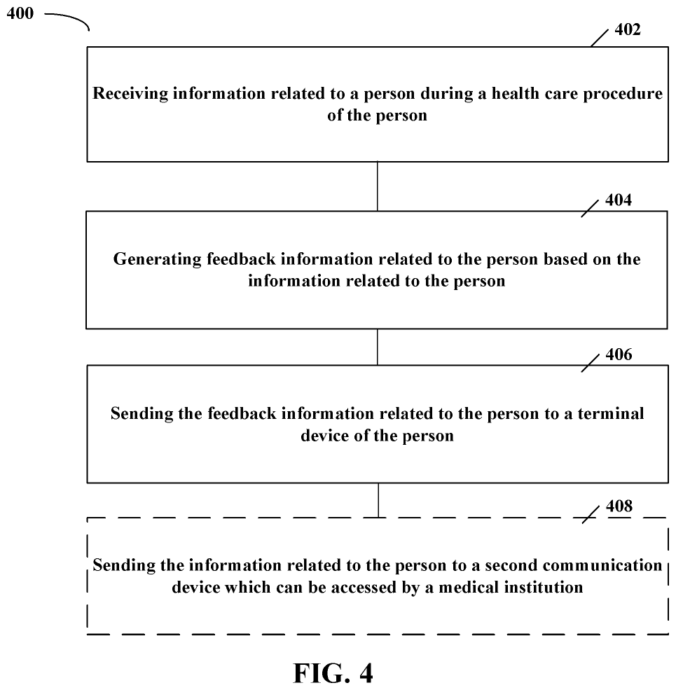

FIG. 4 shows a flowchart of a method 400 according to an embodiment of the present disclosure, which may be performed by an apparatus implemented in or as or communicatively coupled to a first communication device or any other entity having similar functionality. As such, the first communication device may provide means or modules for accomplishing various parts of the method 400 as well as means or modules for accomplishing other processes in conjunction with other components. The first communication device may be located in a medical institution or a cloud computing.

At block 402, the first communication device may receive information related to a person during a health care procedure of the person. For example, the first communication device may receive this information from various devices such as a terminal device of the person and/or a device storing at least a part of this information and/or a device capable of obtaining at least a part of this information.

As used herein, health is care that can help the person (such as patient) get back, keep, or improve abilities that they need for daily life. These abilities may be physical, mental, and/or cognitive (thinking and learning). The person may have lost them because of a disease or injury, or as a side effect from a medical treatment. Rehabilitation can improve the person daily life and functioning. Some of the common causes may include injuries and trauma, including burns, fractures (broken bones), traumatic brain injury, and spinal cord injuries; stroke; severe infections; major surgery; side effects from medical treatments, such as from cancer treatments; certain birth defects and genetic disorders; developmental disabilities; chronic pain, including back and neck pain, etc.

A goal of health is to help the person get abilities back and regain independence. But the specific goals may be different for each person. They may depend on what caused the problem, whether the cause is ongoing or temporary, which abilities the patients lost, and how severe the problem is. For example, a person who has had a stroke may need health to be able to dress or bathe without help. An active person who has had a heart attack may go through cardiac health to try to return to exercising. Someone with a lung disease may get pulmonary health to be able to breathe better and improve their quality of life.

The types of treatments that may be in a treatment plan may include at least one of assistive devices, which are tools, equipment, and products that help the patients with disabilities move and function; cognitive health therapy to help the patients relearn or improve skills such as thinking, learning, memory, planning, and decision making; mental health counseling; music or art therapy to help the patients express feelings, improve thinking, and develop social connections; nutritional counseling; occupational therapy to help the person with his/her daily activities; physical therapy to help his/her strength, mobility, and fitness; recreational therapy to improve his/her emotional well-being through arts and crafts, games, relaxation training, and animal-assisted therapy; speech-language therapy to help with speaking, understanding, reading, writing and swallowing; treatment for pain; vocational health to help the patients build skills for going to school or working at a job, etc.

Depending on the person's needs, he/she may have health in the providers' offices, a hospital, or an inpatient health center.

The information related to the person may include any suitable information which can be used in the health care procedure of the person such as various expressions of the person, activities of the person, monitoring information of the person, living condition of the person, environment information of the person, etc. In an embodiment, the information related to the person may include at least one of a micro expression video clip of the person; a series of body gestures of the person; a shopping record of the person; a sport activity plan of the person; a sport activity record of the person; a health status of the person; a body physical status of the person; a medical record of the person; a work habit of the person; a life habit of the person; diet information of the person; a physical status of the person; a psychological status of the person; nutrition information of the person; and rest information of the person.

The information related to the person may be obtained in various ways. For example, the person and/or his/her nursing staff may input some of the information related to the person (such as the sport activity plan of the person, the health status of the person, the work habit of the person; the life habit of the person, etc.). Some of the information related to the person (such as the micro expression video clip of the person; the series of body gestures of the person, the diet information of the person, the nutrition information of the person; rest information of the person, etc.) may be obtained by various devices such as sensors and wearable devices. Some of the information related to the person (such as the medical record of the person, the shopping record of the person, the sport activity record of the person, etc.) may be obtained from another device storing this information related to the person.

In an embodiment, the health care procedure may comprise at least one of health rehabilitation; health recovery; improvement of health; and strengthening of a level of athletes' competition. The health care procedure may be applied in the following scenarios such as trauma, internal injuries, infectious disease injuries, etc. For example, trauma may include sports injuries (e.g., “muscles and bones”) and normal exercise recovery (e.g., athletes' intense training or recovery after a competition). Internal injuries may include some diseases that affect sports, such as gastrointestinal, cardiovascular and other diseases. Even if it does not affect exercise, it may have some effects in daily life, such as high myopia. Infectious disease injuries may include SARS (Severe acute respiratory syndrome) and COVID-19 (Coronavirus Disease 2019), etc., even recovered patients may have a great physical damage.

At block 404, the first communication device may generate feedback information related to the person based on the information related to the person. The feedback information related to the person may be any suitable information such as information related to health level, suggestion, updated health plan, a request for providing more information, etc.

In an embodiment, the feedback information related to the person may include at least one of a health level of the person; a suggestion; and an updated health plan. The health level of the person may indicate a recovery progress such as how well this person is recovered. The suggestion may include any suitable suggestion(s) such as diet suggestion, rest suggestion, sport activity suggestion, work habit suggestion, life habit suggestion, nutrition suggestion, etc. The updated health plan may include any suitable added, removed, adjusted item(s) comparing with the previous health plan.

The feedback information related to the person may be generated in various ways. For example, it may be generated by the first communication device and/or a health specialist. In an embodiment, the first communication device may generate the feedback information related to the person for example based on a predefined rule/pattern. For example, the predefined rule/pattern may be generated by a health specialist, or may be generated by using machine learning and/or big data technology, or may be generated by using machine learning and/or big data technology and then be adjusted by the health specialist.

In an embodiment, the first communication device may generate the feedback information by providing the information related to the person to a health specialist; and receiving the feedback information related to the person input by the health specialist. For example, the health specialist may generate the feedback information based on the information related to the person and his/her professional knowledge and experience.

In an embodiment, the first communication device may generate the feedback information by generating preliminary feedback information related to the person based on the information related to the person; providing the preliminary feedback information related to the person to a health specialist; and receiving the feedback information related to the person input by the health specialist. For example, the first communication device may generate the preliminary feedback information related to the person based on the information related to the person for example by using a predefined rule/pattern. Then the preliminary feedback information may be provided to the health specialist who may review the preliminary feedback information, amend the preliminary feedback information if required and input the final feedback information to the first communication device.

In an embodiment, the first communication device or the health specialist may generate feedback information related to the person based on the information related to the person by comparing current information related to the person with previous information related to the person and determining the health level of the person based on the comparing result. For example, by comparing the difference between current body gesture and previous body gesture, and referring to the correct and normal body gesture, the first communication device or the health specialist can make a conclusion that how well this person is recovered.

In an embodiment, the first communication device or the health specialist may generate feedback information related to the person based on the information related to the person by determining one or more factors having a strong relationship of the health care procedure based on the information related to at least one quickest recovering person, wherein the at least one quickest recovering person and the person belong to a same or similar health group; determining one or more gaps between the person and the quickest recovering person based on the one or more factors having the strong relationship of the health care procedure and/or an ideal recovering model by historical data; and generating the suggestion based on the one or more gaps. The same or similar health group may mean that people requiring the same or similar health care procedure may belong to the same or similar group. For example, people may be divided in groups for example by a type of disease. For example, based on all information that collected and shared from the quickest recovering person, the first communication device or the health specialist may determine some major factors that have most strong relationship of the health care procedure; analyze the slower recovering person and find out the facts that have big gaps with the quickest recovering person; and provide suggestions with other medical knowledge and provide feasible adjusted health plan to the slower recovering person.

In an embodiment, when there are two or more types of the information related to the person, at least one of the two or more types of the information related to the person may be configured with a weight value. For example, the health specialist such as doctor may configure at least one type of the information related to the person parameters with a weight value based on the specific type of health care procedure of the person. If this person is suffering from physical injury such as legs or arms injuries, then rest may be a more important factor comparing to nutrition. Also, health by a professional trainer is good to the person. Thus, in this example, the health specialist may place more weight value on rest than nutrition. If this person is suffering from disease, then both rest and nutrition may play important roles in his/her health care procedure. Thus, the health specialist may place similar weight values on both rest and nutrition.

In an embodiment, the feedback information related to the person may be authorized by a health specialist. In other embodiments, the feedback information related to the person may not be authorized by the health specialist.

At block 406, the first communication device may send the feedback information related to the person to a terminal device of the person. The terminal device may provide the feedback information to the person who may review the feedback information and then continue or adjust health care procedure based on the feedback information.

At block 408, optionally, the first communication device may send the information related to the person to a second communication device which can be accessed by a medical institution. The health specialist and/or a device of the medical institution can perform studies and researches based on the information that the person shares. This would enable the medical industry to develop a quicker and better health solution.

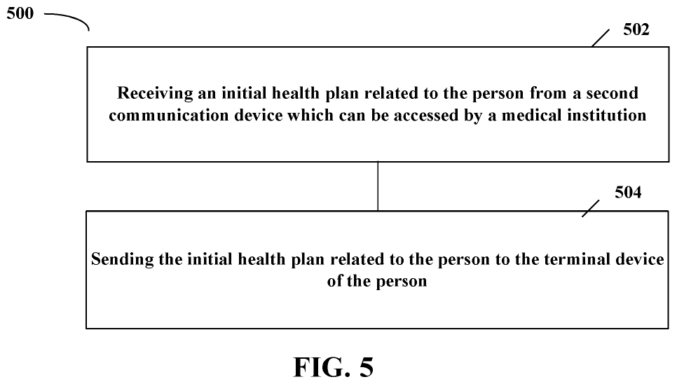

FIG. 5 shows a flowchart of a method 500 according to another embodiment of the present disclosure, which may be performed by an apparatus implemented in or as or communicatively coupled to the first communication device or any other entity having similar functionality. As such, the first communication device may provide means or modules for accomplishing various parts of the method 500 as well as means or modules for accomplishing other processes in conjunction with other components. The first communication device may be located in the medical institution or the cloud computing. For some parts which have been described in the above embodiments, detailed description thereof is omitted here for brevity.

At block 502, the first communication device may receive an initial health plan related to the person from a second communication device which can be accessed by a medical institution. For example, the second communication device may receive the information related to one or more persons belong to the same or similar health group from one or more first communication devices, generate the initial health plan for one or more persons belong to the same or similar health group and send the initial health plan to the first communication device.

At block 504, the first communication device may send the initial health plan related to the person to the terminal device of the person. For example, after therapy of the person for example in the hospital or online, the first communication device may send the initial health plan related to the person to the terminal device of the person.

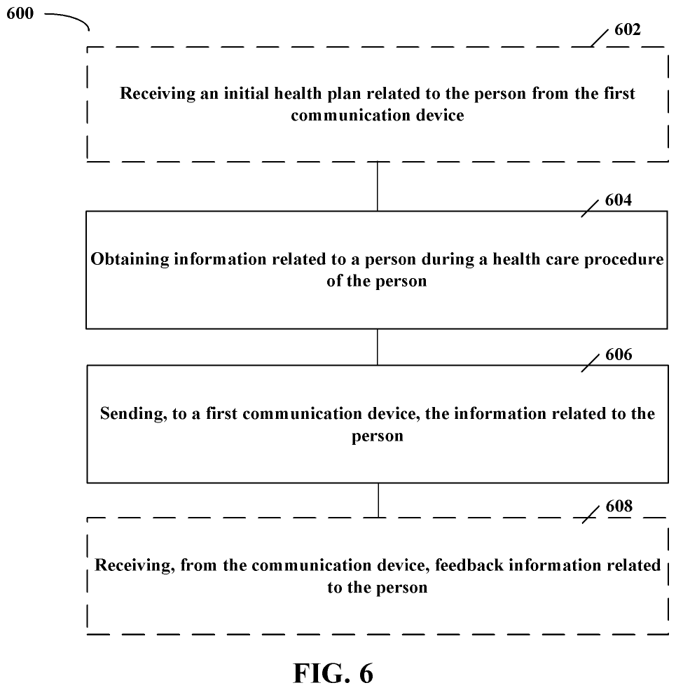

FIG. 6 shows a flowchart of a method 600 according to another embodiment of the present disclosure, which may be performed by an apparatus implemented in or as or communicatively coupled to a third communication device or any other entity having similar functionality. As such, the third communication device may provide means or modules for accomplishing various parts of the method 600 as well as means or modules for accomplishing other processes in conjunction with other components. For some parts which have been described in the above embodiments, detailed description thereof is omitted here for brevity. The third communication device may include one or more communication devices which can obtain the information related to a person during a health care procedure of the person.

At block 602, optionally, the third communication device may receive an initial health plan related to the person from the first communication device. For example, when the third communication device is a terminal device of the person, the third communication device may receive the initial health plan related to the person from the first communication device.

At block 604, the third communication device may obtain information related to a person during a health care procedure of the person. The third communication device may obtain this information in various ways. For example, the person may input some information related to the person into the third communication device. The third communication device may obtain some information related to the person by itself for example by using various sensors of the third communication device. The third communication device may obtain some information related to the person from another device which can obtain and/or store this information.

At block 606, the third communication device may send, to the first communication device, the information related to the person.

At block 608, optionally, the third communication device may receive, from the communication device, feedback information related to the person. For example, when the third communication device is a terminal device of the person, the third communication device may receive, from the communication device, the feedback information related to the person.

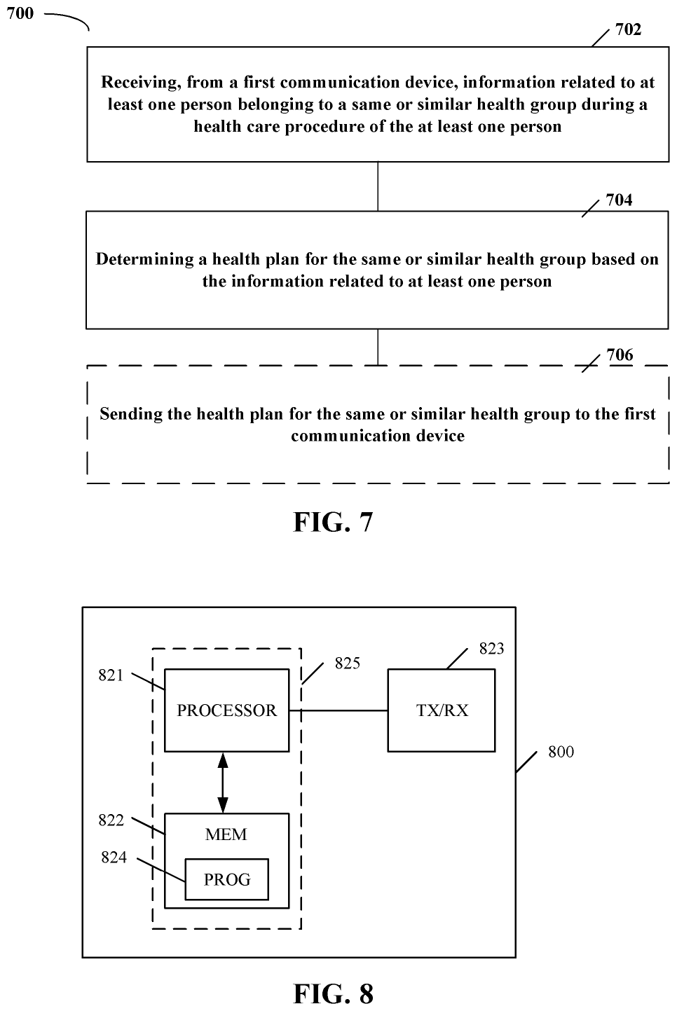

FIG. 7 shows a flowchart of a method 700 according to another embodiment of the present disclosure, which may be performed by an apparatus implemented in or as or communicatively coupled to a second communication device or any other entity having similar functionality. As such, the second communication device may provide means or modules for accomplishing various parts of the method 700 as well as means or modules for accomplishing other processes in conjunction with other components. For some parts which have been described in the above embodiments, detailed description thereof is omitted here for brevity. The second communication device may be located in in a medical institution or a cloud computing.

At block 702, the second communication device may receive, from a first communication device, information related to at least one person belonging to a same or similar health group during a health care procedure of the at least one person. For example, the first communication device may send the information related to the person to the second communication device at block 408 of FIG. 4, then the second communication device may receive, from the first communication device, this information.

At block 704, the second communication device may determine a health plan for the same or similar health group based on the information related to at least one person. For example, the second communication device may determine the health plan for the same or similar health group based on the information related to at least one person by using machine learning and/or big data technology.

In an embodiment, the information related to the at least one person may include at least one of a micro expression video clip of the at least one person; a series of body gestures of the at least one person; a shopping record of the at least one person; a sport activity plan of the at least one person; a sport activity record of the at least one person; a health status of the at least one person; a body physical status of the at least one person; a medical record of the at least one person; a work habit of the at least one person; a life habit of the at least one person; diet information of the at least one person; a physical status of the at least one person; a psychological status of the at least one person; nutrition information of the at least one person; and rest information of the at least one person.

In an embodiment, when there are two or more types of the information related to the at least one person, at least one of the two or more types of the information related to the at least one person may be configured with a weight value.

At block 706, optionally, the second communication device may send the health plan for the same or similar health group to the first communication device.

In an embodiment, the proposed solution may utilize the camera installed in the public areas, such as streets, city halls, supermarkets, etc.; the VR/AR glasses that the patient is wearing; the medical record database that the hospital owned, to obtain the information related to a person during a health care procedure of the person.

In an embodiment, with the information that obtained or stored in various communication devices such as the above facilities, the proposed solution may categorize all persons such as patients into different groups in the criteria that defined by a health specialist such as doctor. For example, each group may stand for a dedicated disease, and under each group, there can be a series of sub-groups which are identified by genders, ages, etc.

In an embodiment, people who belong to different sub-groups may be treated medically in their specified health methods to reach the maximum health as quickly as possible.

In order to make reasonable sub-groups, the proposed solution may apply an algorithm to do that in assistance by a health specialist such as doctor. Based on the progress and status of each person's health, the health specialist can also update the algorithm manually.

In an embodiment, the proposed solution may adjust the health care procedure that dedicated for a certain person by utilizing the existing devices that he/she has. Also, the expected data may be transferred to the first communication device operated by the health specialist in a daily basis rather than reporting details initiatively. In this way, the person may have much better experience comparing to visiting the health specialist in person which might need to wait in a long queue.

In an embodiment, the health specialist can also free his/her time to evaluate one or more persons' health status and provide specified improvement proposals to anyone, for example the persons who is in relative slow recovery progress. This may be useful for some areas where major medical resources are unbalanced distributed. The health specialist and famous hospitals are usually in big cities where many patients are, so health specialists are rather overloaded by works. If assisted with the proposed solution, the health specialist can pay more attention to persons in both health care procedure and treatment.

In an embodiment, the medical institution can do extra studies and researches based on data that many persons shares. This may enable the medical industry to develop a quicker and better health solution.

In an embodiment, the proposed solution may benefit various parties such as the person who requires health care, the health specialist and the medical institution in a combined and integrated way.

The various blocks or step shown in FIGS. 4-7 may be viewed as method steps, and/or as operations that result from operation of computer program code, and/or as a plurality of coupled logic circuit elements constructed to carry out the associated function(s). The schematic flow chart diagrams described above are generally set forth as logical flow chart diagrams. As such, the depicted order and labeled steps are indicative of specific embodiments of the presented methods. Other steps and methods may be conceived that are equivalent in function, logic, or effect to one or more steps, or portions thereof, of the illustrated methods. Additionally, the order in which a particular method occurs may or may not strictly adhere to the order of the corresponding steps shown.

Embodiments herein afford many advantages, of which a non-exhaustive list of examples follows. Some embodiments herein can improve the person's health care procedure. For example, the person may not be suffered too long if the person's health care procedure is in a wrong way (which may happen in some areas such as remote and backward areas). Some embodiments herein can enable the health specialist's efforts on the consequence treatment based on the health status to be significantly reduced. Some embodiments herein can enable the health specialist to pay more efforts on study more cases or improve the medical professional skills that may bring much more benefit to the person who requires health care. In some embodiments herein, the information between the person and the health specialist is more transparent and objective with less interference information. Some embodiments herein can utilize various facilities such as camera along the street, video captured by phones, online shopping records, sports exercises plans and records, etc. to create a database for the person who requires health care. The embodiments herein are not limited to the features and advantages mentioned above. A person skilled in the art will recognize additional features and advantages upon reading the following detailed description.

FIG. 8 is a block diagram showing an apparatus suitable for practicing some embodiments of the disclosure. For example, any one of the first communication device, the second communication device and the third communication device described above may be implemented as or through the apparatus 800.

The apparatus 800 comprises at least one processor 821, such as a digital processor (DP), and at least one memory (MEM) 822 coupled to the processor 821. The apparatus 820 may further comprise a transmitter TX and receiver RX 823 coupled to the processor 821. The MEM 822 stores a program (PROG) 824. The PROG 824 may include instructions that, when executed on the associated processor 821, enable the apparatus 820 to operate in accordance with the embodiments of the present disclosure. A combination of the at least one processor 821 and the at least one MEM 822 may form processing means 825 adapted to implement various embodiments of the present disclosure.

Various embodiments of the present disclosure may be implemented by computer program executable by one or more of the processor 821, software, firmware, hardware or in a combination thereof.

The MEM 822 may be of any type suitable to the local technical environment and may be implemented using any suitable data storage technology, such as semiconductor based memory devices, magnetic memory devices and systems, optical memory devices and systems, fixed memories and removable memories, as non-limiting examples.

The processor 821 may be of any type suitable to the local technical environment, and may include one or more of general purpose computers, special purpose computers, microprocessors, digital signal processors DSPs and processors based on multicore processor architecture, as non-limiting examples.

In an embodiment where the apparatus is implemented as or at the first communication device, the memory 822 contains instructions executable by the processor 821, whereby the first communication device operates according to any of the methods as described in reference to FIGS. 4-5.

In an embodiment where the apparatus is implemented as or at the third communication device, the memory 822 contains instructions executable by the processor 821, whereby the third communication device operates according to the method 600 as described in reference to FIG. 6.

In an embodiment where the apparatus is implemented as or at the second communication device, the memory 822 contains instructions executable by the processor 821, whereby the second communication device operates according to the method 700 as described in reference to FIG. 7.

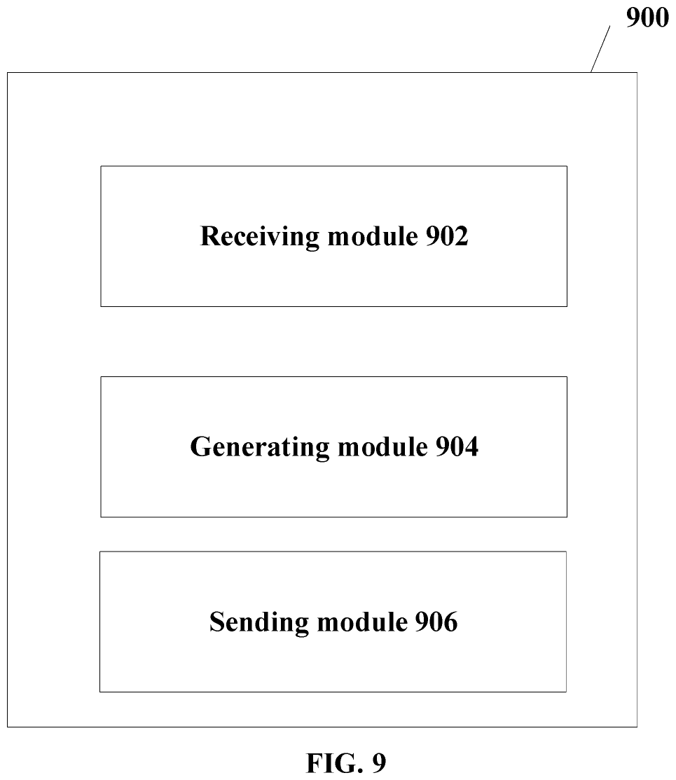

FIG. 9 is a block diagram showing a first communication device according to an embodiment of the disclosure. As shown, the first communication device 900 comprises a receiving module 902, a generating module 904 and a sending module 906. The receiving module 902 may be configured to receive information related to a person during a health care procedure of the person. The generating module 904 may be configured to generate feedback information related to the person based on the information related to the person. The sending module 906 may be configured to send the feedback information related to the person to a terminal device of the person.

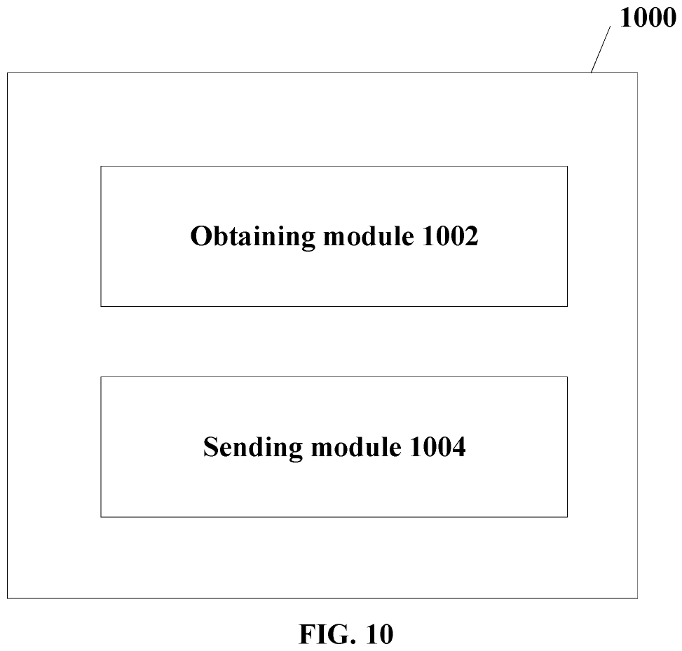

FIG. 10 is a block diagram showing a third communication device according to an embodiment of the disclosure. As shown, the third communication device 1000 comprises a obtaining module 1002 and a sending module 1004. The obtaining module 1002 may be configured to obtain information related to a person during a health care procedure of the person. The sending module 1004 may be configured to send, to a first communication device, the information related to the person.

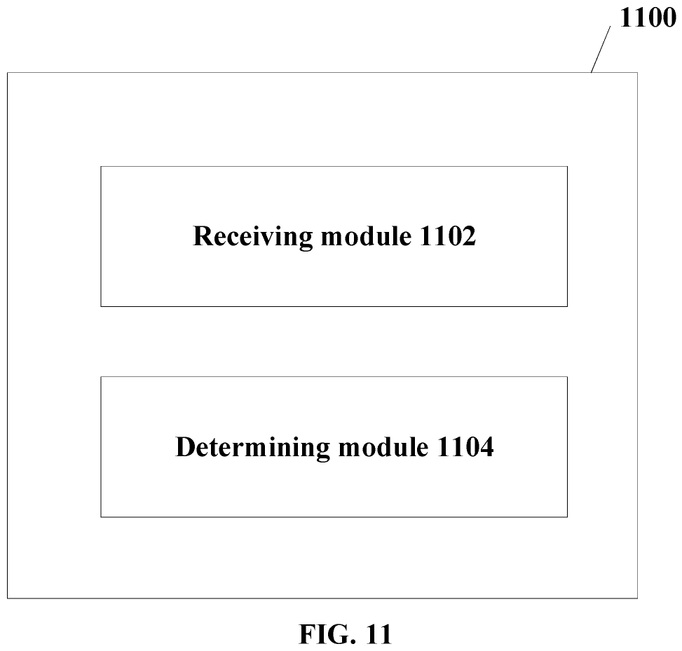

FIG. 11 is a block diagram showing a second communication device according to an embodiment of the disclosure. As shown, the second communication device 1100 comprises a receiving module 1102 and a determining module 1104. The receiving module 1102 may be configured to receive, from a first communication device, information related to at least one person belonging to a same or similar health group during a health care procedure of the at least one person. The determining module 1104 may be configured to determine a health plan for the same or similar health group based on the information related to at least one person.

According to an aspect of the disclosure it is provided a computer program product being tangibly stored on a computer readable storage medium and including instructions which, when executed on at least one processor, cause the at least one processor to carry out any of the methods as described above.

According to an aspect of the disclosure it is provided a computer-readable storage medium storing instructions which when executed by at least one processor, cause the at least one processor to carry out any of the methods as described above.

The term unit or module may have conventional meaning in the field of electronics, electrical devices and/or electronic devices and may include, for example, electrical and/or electronic circuitry, devices, modules, processors, memories, logic solid state and/or discrete devices, computer programs or instructions for carrying out respective tasks, procedures, computations, outputs, and/or displaying functions, and so on, as such as those that are described herein.

In addition, the present disclosure may also provide a carrier containing the computer program as mentioned above, wherein the carrier is one of an electronic signal, optical signal, radio signal, or computer readable storage medium. The computer readable storage medium can be, for example, an optical compact disk or an electronic memory device like a RAM (random access memory), a ROM (read only memory), Flash memory, magnetic tape, CD-ROM, DVD, Blue-ray disc and the like.

The techniques described herein may be implemented by various means so that an apparatus implementing one or more functions of a corresponding apparatus described with an embodiment comprises not only prior art means, but also means for implementing the one or more functions of the corresponding apparatus described with the embodiment and it may comprise separate means for each separate function or means that may be configured to perform two or more functions. For example, these techniques may be implemented in hardware (one or more apparatuses), firmware (one or more apparatuses), software (one or more modules), or combinations thereof. For a firmware or software, implementation may be made through modules (e.g., procedures, functions, and so on) that perform the functions described herein.

Exemplary embodiments herein have been described above with reference to block diagrams and flowchart illustrations of methods and apparatuses. It will be understood that each block of the block diagrams and flowchart illustrations, and combinations of blocks in the block diagrams and flowchart illustrations, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

Further, while operations are depicted in a particular order, this should not be understood as requiring that such operations be performed in the particular order shown or in sequential order, or that all illustrated operations be performed, to achieve desirable results. In certain circumstances, multitasking and parallel processing may be advantageous. Likewise, while several specific implementation details are contained in the above discussions, these should not be construed as limitations on the scope of the subject matter described herein, but rather as descriptions of features that may be specific to particular embodiments. Certain features that are described in the context of separate embodiments may also be implemented in combination in a single embodiment. Conversely, various features that are described in the context of a single embodiment may also be implemented in multiple embodiments separately or in any suitable sub-combination.

While this specification contains many specific implementation details, these should not be construed as limitations on the scope of any implementation or of what may be claimed, but rather as descriptions of features that may be specific to particular embodiments of particular implementations. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

It will be obvious to a person skilled in the art that, as the technology advances, the inventive concept can be implemented in various ways. The above described embodiments are given for describing rather than limiting the disclosure, and it is to be understood that modifications and variations may be resorted to without departing from the spirit and scope of the disclosure as those skilled in the art readily understand. Such modifications and variations are considered to be within the scope of the disclosure and the appended claims. The protection scope of the disclosure is defined by the accompanying claims.

1. A method at a first communication device, comprising:
receiving information related to a person during a health care procedure of the person;
generating feedback information related to the person based on the information related to the person; and
sending the feedback information related to the person to a terminal device of the person.

2. The method according to claim 1, wherein the feedback information related to the person is authorized by a health specialist.

3. The method according to claim 1, wherein generating feedback information related to the person based on the information related to the person comprises:
providing the information related to the person to a health specialist; and receiving the feedback information related to the person input by the health specialist.

4. The method according to claim 1, wherein generating feedback information related to the person based on the information related to the person comprises:
generating preliminary feedback information related to the person based on the information related to the person;
providing the preliminary feedback information related to the person to a health specialist; and
receiving the feedback information related to the person input by the health specialist.

5. The method according to claim 1, wherein the feedback information related to the person includes at least one of:
a health level of the person;
a suggestion; and
an updated health plan.

6. The method according to claim 5, wherein generating feedback information related to the person based on the information related to the person comprises:
comparing current information related to the person with previous information related to the person; and
determining the health level of the person based on the comparing result.

7. The method according to claim 5, wherein generating feedback information related to the person based on the information related to the person comprises:
determining one or more factors having a strong relationship of the health care procedure based on the information related to at least one quickest recovering person, wherein the at least one quickest recovering person and the person belong to a same or similar health group;
determining one or more gaps between the person and the quickest recovering person based on the one or more factors having the strong relationship of the health care procedure and/or an ideal recovering model by historical data; and
generating the suggestion based on the one or more gaps.

8. The method according to claim 1, wherein the information related to the person includes at least one of:
a micro expression video clip of the person;
a series of body gestures of the person;
a shopping record of the person;
a sport activity plan of the person;
a sport activity record of the person;
a health status of the person;
a body physical status of the person;
a medical record of the person;
a work habit of the person;
a life habit of the person;
diet information of the person;
a physical status of the person;
a psychological status of the person;
nutrition information of the person; and
rest information of the person.

9. The method according to claim 1, wherein when there are two or more types of the information related to the person, at least one of the two or more types of the information related to the person is configured with a weight value.

10. The method according to claim 1, further comprising:
sending the information related to the person to a second communication device which can be accessed by a medical institution.

11. The method according to claim 1, further comprising:
receiving an initial health plan related to the person from a second communication device which can be accessed by a medical institution; and
sending the initial health plan related to the person to the terminal device of the person.

12. The method according to claim 1, wherein the health care procedure comprises at least one of:
health rehabilitation;
health recovery;
improvement of health; and
strengthening of a level of athletes' competition.

13. A method at a third communication device, comprising:
obtaining information related to a person during a health care procedure of the person; and
sending, to a first communication device, the information related to the person.

14. The method according to claim 13, further comprising:
receiving, from the first communication device, feedback information related to the person.

15. The method according to claim 14, wherein the feedback information related to the person is authorized by a health specialist.

16. The method according to claim 14 wherein the feedback information related to the person includes at least one of:
a health level of the person;
a suggestion; and
an updated health plan.

17. The method according to claim 14, further comprising:
receiving an initial health plan related to the person from the first communication device.

18. The method according to claim 13, wherein the information related to the person includes at least one of:
a micro expression video clip of the person;
a series of body gestures of the person;
a shopping record of the person;
a sport activity plan of the person;
a sport activity record of the person;
a health status of the person;
a body physical status of the person;
a medical record of the person;
a work habit of the person;
a life habit of the person;
diet information of the person;
a physical status of the person;
a psychological status of the person;
nutrition information of the person; and
rest information of the person.

19. The method according to claim 13, wherein the health care procedure comprises at least one of:
health rehabilitation;
health recovery;
improvement of health; and
strengthening of a level of athletes' competition.

20. A method at a second communication device, comprising:
receiving, from a first communication device, information related to at least one person belonging to a same or similar health group during a health care procedure of the at least one person; and
determining a health plan for the same or similar health group based on the information related to at least one person.

21.-32. (canceled)
